SEQUENCE LISTING (4 sequences)

SEQ ID NO: 1 - DNA, 17 bp, Artificial
  Synthetic
acgtacgtaggggggga

SEQ ID NO: 2 - DNA, 17 bp, artificial
  Synthetic
agggggggacgtacgta

SEQ ID NO: 3 - DNA, 25 bp, Artificial
  Synthetic
acgtacgtagggggggacgtacgta

SEQ ID NO: 4 - DNA, 25 bp, Artificial
  Synthetic
agggggggacgtacgtaggggggga

(19) United States
(12) Patent Application Publication
(10) Pub. No.: US 2007/0212710 A1
(43) Pub. Date: Sep. 13, 2007

(54) COMPETITIVE HYBRIDIZATION OF DNA PROBES AND METHOD OF USING THE SAME

(75) Inventor: Michael P. Mittmann, Palo Alto, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/619,143

(22) Filed: Jan. 2, 2007

Related U.S. Application Data
(60) Provisional application No. 60/754,667, filed on Dec. 30, 2005.

(51) Int. Cl. C12Q 1/68; C07H 21/04

(52) U.S. Cl. 435/6; 536/24.3

(57) ABSTRACT

Methods are presented, in accordance for the present invention, for determining the length of a target probe. The methods have the steps of designing a first hybridization probe having a nucleic acid sequence, a portion of which overlaps with the nucleic acid sequence of a second hybridization probe, designing a second hybridization probe having a nucleic acid sequence, a portion of which overlaps with the nucleic acid sequence of the first hybridization probe, designing a target probe having the nucleic acid sequences of both the first and second hybridization probe and affixing the target probe to a solid support, labeling one of the first and second hybridization probes, but not both, and contacting simultaneously the first and second probes to the target probe, and detecting and quantifying the signal intensity ration between the labeled and non-labeled probes, whereby said ration indicating whether the target probe synthesis has reached full length.

### FIELD OF THE INVENTION

The present invention relates generally to a method for assessing the results of competitive hybridization between polynucleotide sequences. More particularly, the present methods use competitive hybridization and differential labeling to discriminate between synthesized full-length probes and non-full length probes. The methods of the present invention will find broad application in the analysis of probe quality for microarray technology.

### BACKGROUND

A microarray, nucleotide, oligonucleotide array, or genome chip, may include hundreds of thousands of nucleic acid probes. Probes may include a known nucleic acid sequence which may be used to recognize longer, unknown nucleic acid sequences. The recognition of sample nucleic acid by the set of nucleic acid probes on a solid support such as a glass wafer (or chip) takes place through the mechanism of nucleic acid hybridization. When a nucleic acid sample hybridizes with an array of nucleic acid probes, the sample will bind to those probes that are complementary to a target nucleic acid sequence. By evaluating to which probes the sample nucleic acid hybridizes more strongly, it can be determined whether a known sequence of DNA is present or not in the sample nucleic acid.

One of the problems one skilled in the art face in constructing nucleic acid probes is that in each synthesis step there is a possibility that the synthesis may terminate before the probes reach their full lengths. Premature termination of probe synthesis may result in a mixture of probes with different lengths. For example, in the synthesis of 25-mer probes, premature termination may result in a population of probes where 3% of the probes may be 15-mers, 3% being 16-mers, 3% being 17-mers, and 10% being 25-mers. Use of such a probe population in the construction of gene chips will inevitably compromise the quality of the chips to be made.

In the work leading up to the present invention, the inventor developed a full length probe detection system which applies competitive nucleic acid hybridization and differential labeling to produce a method capable of discriminating between full-length probes and non full-length length probes. The present invention provides a method for determining the length of a probe at the nucleotide level by quantifying signal intensity ratio of labeled to non-labeled hybridization probes immobilized on the target probes. The method of the present invention is also capable of being multiplexed and automated.

### SUMMARY OF THE INVENTION

The present invention provides a method for determining the quality of full-length probe synthesis using differential labeling and competitive nucleic acid hybridization.

According to an embodiment of the method, a first hybridization probe comprising a nucleic acid sequence is designed, a portion of which overlaps with the nucleic acid sequence of a second hybridization probe. A second hybridization probe comprising a nucleic acid sequence is designed such that a portion of its nucleic acid sequence also overlaps with that of the first hybridization probe. A target probe comprising the nucleic sequences of both the first and second hybridization probes or either of the first and second hybridization probes is designed and subsequently affixed to a solid support. After one of the first and second hybridization probes, but not both, are labeled, the first and second hybridization probes are contacted simultaneously with the target probes affixed on the solid support for hybridization. Once immobilized, the immobilized target nucleic acids are then detected by the detectable label attached to the hybridization probes. The signal intensity ratio of the labeled to the non-label probes indicates whether the target probes are full length probes.

The assay of the present invention may also be readily adapted for quality control measurement of probes synthesized for microarray construction.

### DETAILED DESCRIPTION OF THE INVENTION

The present invention relates to a rapid and efficient hybridization assay for detecting and accurately quantifying full length target nucleic acid sequences. According to the method of the present invention, two hybridization probes are used which hybridize to the same sequence of a target nucleic acid. By designing the hybridization assay such that the two hybridization probes containing overlapping sequences are hybridized to a target probe whose nucleic acid sequences consist of both the nucleic acid sequences of the two hybridization probes or that of one of the hybridization probes, the inventor has developed a method of determining the length of target probes obtained from different synthesis steps.

In a preferred embodiment, the hybridization assay is used to detect one single nucleotide difference in a synthesized probe.

According to the assay of the present invention, a pair of hybridization probes is first obtained. The first hybridization probe comprises a nucleic acid sequence that may have a functional length of up to about 16, 17, 18, 19, 20, 21, 22, 23, 24, 25 and 50 nucleotides. The first hybridization probe may comprise a nucleic acid sequence represented by the formula:
X-Y

wherein X and Y each represent a portion of the nucleic acid sequence of the first hybridization probe.

The second hybridization probe comprises a nucleic acid sequence that may also have a functional length of up to about 16, 17, 18, 19, 20, 21, 22, 23, 24, 25 and 50 nucleotides. The second hybridization probe may comprise a nucleic acid sequence represented by the formula:
Y-X

wherein X and Y have the same meaning above. Thus, the second hybridization probe has similar predicted thermodynamic properties as the first hybridization probe. However, the second hybridization probe differs from the first hybridization probe in that its nucleic acid sequence is in reverse order.

The target probe, i.e., the probe whose length is to be tested, is a combination of the sequences of the first and second hybridization probes. The target probe therefore comprises a nucleic acid sequence that can be represented by the formula: X-Y-X or Y-X-Y, wherein X and Y have the same meaning above. The target probe may further be represented by the formulas U-X-Y, U-Y-X, X-Y-U or Y-X-U, wherein X and Y have the same meaning as discussed above and U represents some non-matching sequence. The nucleic acid sequence of the target probe may have a functional length of up to about 20, 21, 22, 23, 24, 25, 30, 35, 40, 50 and 100 nucleotides. It should be understood that the functional length for the first and second hybridization probes and the target probe set forth above is only a matter of choice and that any length may be used, for instance, any number of nucleotides from about 10 to about 100 or more. It should also be understood that the present method is not limited to two hybridization probes. Two or more hybridization probes may also be employed to hybridize to the target probe with each probe labeled but with a different label, for instance, two different fluorescent labels.

The target probes are then affixed to a solid support. Any solid support to which nucleic acid be attached may be used in the present invention including wafers, chips and beads sets. Examples of suitable solid support materials include, but are not limited to, porous substrates, non-porous substrates, metals, silicates such as glass and silica gel, cellulose and nitrocellulose papers, nylon, polymers such as polystyrene, polymethacrylate, plastics, latex, rubber, and fluorocarbon resins such as TEFLON™.

The solid support material may be used in a wide variety of shapes including, but not limited to three-dimensional surfaces, planar surfaces such as slides, and beads. Slides provide several functional advantages and thus are a preferred form of solid support. Slides can be readily used with any chromosome organization. Due to their flat surface, probe and hybridization reagents can be minimized using glass slides. Slides also enable the targeted application of reagents, are easy to keep at a constant temperature, are easy to wash and facilitate the direct visualization of RNA and/or DNA immobilized on the solid support. Removal of RNA and/or DNA immobilized on the solid support is also facilitated using slides. It is estimated that a standard microscope glass slide can contain 50,000 to 100,000, 500,000, 1,000,000 or more cells worth of DNA. Beads, such as BioMag Strepavidin magnetic beads are another preferred form of solid support.

After the target probes are fixed to the solid support, one of the first and second hybridization probes, but not both, is labeled with an analytically detectable marker such that a population of either the first or second hybridization probe becomes labeled probes. Any analytically detectable label that can be attached to or incorporated into a hybridization probe may be used in the present invention. An analytically detectable marker refers to any molecule, moiety or atom which can be analytically detected and quantified. Methods for detecting analytically detectable markers include, but are not limited to, radioactivity, fluorescence, absorbance, mass spectroscopy, EPR, NMR, XRF, luminescence and phosphorescence. For example, any radiolabel which provides an adequate signal and a sufficient half-life may be used as a detectable marker. Commonly used radioisotopes include ³H, ¹⁴C, ³²P and ¹²⁵I. In a preferred embodiment, ¹⁴C is used as the detectable marker and is detected by accelerator mass spectroscopy (AMS). ¹⁴C is preferred because of its exceptionally long half-life and because of the very high sensitivity of AMS for detecting ¹⁴C isotopes. Other isotopes that may be detected using AMS include, but are not limited to, ³H, ¹²⁵I, ⁴¹Ca, ⁶³Ni and ³⁶CI.

Fluorescent molecules, such as fluorescein and its derivatives, rhodamine and its derivatives, dansyl, umbeliferone and acridimium, and chemiluminescent molecules such as luciferin and 2,3-dihydrophthalazinediones may also be used as detectable markers. Molecules which bind to an analytically detectable marker may also be covalently attached to or incorporated into hybridization probe, for example, as taught by McGall et al., U.S. Pat. Nos. 6,965,020, 6,864,059, 6,844,433, which are incorporated herein by reference. In such instances, the hybridization probe is detected by adding an analytically detectable marker which specifically binds to the probe, thereby enabling detection of the probe. Examples of such molecules and their analytically detectable counterparts include biotin and either fluorescent or chemiluminescent avidin. Antibodies that bind to an analytically detectable antigen may also be used as a detectable marker. The detectable marker may also be a molecule which, when subjected to chemical or enzymatic modification, becomes analytically detectable such as those disclosed in Leary, et al., Proc. Natl. Acad. Sci. (U.S.A.). 80:4045-4049 (1983) which is incorporated herein by reference. Other examples of suitable detectable markers include protein binding sequences which can be detected by binding proteins, such as those disclosed in U.S. Pat. No. 4,556,643 which is incorporated herein by reference. As discussed herein, the nucleic acid sequence employed in the first and/or second hybridization probe may function as a detectable marker where the bases forming the nucleic acid sequence are quantified using techniques known in the art.

The labeled first hybridization probe and unlabeled second hybridization probe (or unlabeled first hybridization probe and labeled second hybridization probe) are simultaneously contacted with the target sequences affixed on a solid support. The first and second hybridization probes may hybridize to separate and distinct portions of the target sequence or, may hybridize to overlapping portions of the target sequence. The first and second hybridization probes are only limited in that the first and second probes each include a nucleic acid sequence that is specific to a portion of the target sequence but common to another portion of the target sequence, thereby enabling both hybridization probes to simultaneously hybridize to the target sequence.

The hybridization assay of the present invention utilizes the fact that hybridization probes do not perfectly and stoichiometrically hybridize to a target sequence such that only a single hybridization probe binds to a given sequence. Rather, the actual hybridization of a hybridization probe to a target sequence is generally imperfect such that a series of hybridization probes partially hybridize to the same target sequence to which the hybridization probe is complementary. This is particularly true when hybridization probes having a long sequence of nucleic acids are used.

The hybridization assay of the present invention is designed to take advantage of the imperfect, non-stoichiometric hybridization of hybridization probes by utilizing a competitive hybridization scheme in order to detect the length of a target probe of nucleic acids. More specifically, the assay presupposes that the hybridization probes will be imperfect and non-stoichiometric in nature and employs a pair of hybridization probes in which the hybridization probes compete to hybridize to the same target sequence.

Competitive hybridization allows the first and second hybridization probes to hybridize equally to target probes if the target probes are in full length. This is because full-length target probes comprise a sequence that matches both the sequences of the first and second hybridization probes. For example, if the target probe is a full-length probe having the sequence X-Y-X, both the first hybridization probe X-Y and second hybridization probe Y-X can hybridize equally to the target probe as both hybridization probes contain nucleotides that fully match those in the target probes. The signal intensity ratio between the labeled and non-labeled hybridization probes immobilized on the solid support would be equal to 1:1 as both probes have a 50% chance to hybridize to the target sequences.

However, if the target probe is less than full length at one of its termini, the two hybridization probes would not compete equally in their binding to the target probes. For example, suppose again that the target probe is X-Y-X only that there are a few nucleotides missing in X at the right terminus of the sequence. The first hybridization probe X-Y probe would still bind strongly to the target probe as all its nucleotides match those in the target sequence. The second hybridization probe Y-X, however, would not hybridize as strongly to the target probe because not all of its nucleotides match those in the target sequence due to the missing nucleotides in X at the right terminus of the target sequence. As a result, probe Y-X would not effectively compete with probe X-Y in its binding to the target sequence and more probe Y-X would be displaced by probe X-Y. If probe X-Y is labeled whereas probe Y-X is unlabeled, a higher signal intensity ratio of probe X-Y to probe Y-X would be observed for the probes hybridized to the target sequence. A more detailed discussion of competitive hybridization between the hybridization probes and the target probe is provided in the Example bellow.

The competitive nature of the hybridization assay of the present invention provides unusual control over the sensitivity of the hybridization assay. It also provides a faster, more accurate and more sensitive method for detecting and quantifying nucleic acid sequences.

The hybridization probes and target probes may include RNA or DNA sequences or mixtures of RNA and DNA sequences such that the complementary nucleic acid sequences formed between the hybridization probes and the target sequence may be two DNA sequences, two RNA sequences or an RNA and a DNA sequence.

The amount of the first hybridization probe relative to the second hybridization probe used in the hybridization is approximately equal. As a result, the first and second hybridization probes have the same relative concentration. By keeping the relative concentration of the first hybridization probe to the second hybridization probe constant, the proportion of hybridization probes hybridizing to the target sequence from the first and second hybridization probes should correlate to the amount of the first and second hybridization probes that are used.

The ratio between the first and second fractions of hybridization probes may be used to control the sensitivity of the hybridization assay. According to the present invention, the first and second hybridization probes are simultaneously contacted with the target probe such that the two fractions of hybridization probes competitively hybridize to the target sequence. By causing the first and second fractions of hybridization probes to undergo competitive hybridization, and because the first and second hybridization probe fractions contain the same relative concentrations of first and second hybridization probes, the number of first and second hybridization probes that hybridize to the target sequence from each fraction can be controlled as a function of the ratio between the first fraction and the second fraction in the mixture of hybridization probes employed to perform the assay. For example, by using a higher ratio of second fraction probes to first fraction probes, a greater number of second hybridization probes will hybridize to the target sequence. Assuming the second hybridization probe is labeled, a greater number of detectable labels will be immobilized to indicate the presence of the second hybridization probe. This enables one to control the amount of detectable marker that becomes attached to the target sequence, thereby providing the user of the present assay with control over the amount of detectable marker that becomes attached to the target sequence. Accordingly, one is able to increase or decrease the sensitivity of the assay of the present invention by increasing or decreasing the ratio of the second hybridization probes to the first hybridization probes.

It is preferred that the ratio between the first hybridization probes and the second hybridization probes is about 1:1.

The immobilized hybridization probes are then separated from any nonimmobilized hybridization probes. Separation of the immobilized nucleic acids from non-immobilized nucleic acids may be accomplished by a variety of methods known in the art including, but not limited to, centrifugation, filtration, magnetic separation, chemical separation and washing.

After the immobilized target sequences have been separated from any non-immobilized nucleic acids, the immobilized sequences are analyzed for the presence of a detectable marker. The quantity of a target sequence in a sample can then be readily determined by quantifying the detectable marker.

Once any nucleic acids and hybridization probes that are not immobilized to the solid support have been removed, the presence or absence of the detectable marker attached to the hybridization probes is detected in order to quantify the target sequence. The detection and quantification of the detectable marker can be performed using a variety of methods, depending upon the particular hybridization probes and detectable markers employed.

The detectable marker may be detected by a variety of methods known in the art, depending on the particular detectable marker employed. For example, AMS may be used when the detectable marker is a radioisotope such as ¹⁴C, liquid scintillation may be used when the detectable marker is tritiated thymidine and standard fluorescence or spectroscopic methods may be used when the detectable marker is a fluorescent molecule or the DNA itself.

The quantity of the target nucleic acid sequence that is present may be determined based on the signal generated from the detectable marker using a calibration curve. The calibration curve may be formed by analyzing a serial dilution of a sample of nucleic acids having a known concentration of the target sequence. For example, a calibration curve may be generated by analyzing a series of known amounts of target sequences, the concentration of which can be determined in the process of their synthesis. Alternatively, the amount of nucleic acid material may be analyzed according to the method of the present invention and according to a method known in the art for quantifying the target nucleic acid sequence. Alternative methods for generating a calibration curve are within the level of skill in the art and may be used in conjunction with the method of the present invention.

The following examples set forth the method for detecting the length of a target probe according to the present invention. Further objectives and advantages of the present invention other than those set forth above will become apparent from the examples which are not intended to limit the scope of the present invention.

### EXAMPLE

The embodiments of the present invention may be further elucidated by the following example.

First, a first hybridization probe, designated as “A” is designed. Probe A is a 17-mer having the nucleic acid sequence of ACGTACGTAGGGGGGGA (SEQ ID NO. 1). Next, a second hybridization probe, designated as “B” is designed. Probe B is also a 17-mer, having the nucleic acid sequence of AGGGGGGGACGTACGTA (SEQ ID NO. 2). It is worth noting that the sequences of the first and second probes overlap with each other (see the sequences underlined and in bold).

The target sequence is designed such that, in one aspect, its sequence comprises the nucleic acid sequences of the first and second hybridization probes. Thus, the target probe may be represented by the formula AB wherein AB is a 25-mer having the sequence of ACGTACGTAGGGGGGGACGTACGTA (SEQ ID NO 3), or the target probe may be represented by the formula BA wherein BA is a 25-mer having the sequence of AGGGGGGGACGTACGTAGGGGGGGA (SEQ ID NO. 4). For purposes of illustration, only hybridization between the target sequence AB and probes A and B is discussed. The mechanisms of hybridization between probes A and B and the target sequence BA, UA, AU, UB, BU wherein U represents some non-matching sequence would be the same.

In order to determine whether sequence AB has been synthesized to its full length, i.e., a 25-mer, sequence AB is affixed to a solid support and hybridized simultaneously with the first hybridization probe, Probe A, which is labeled and the second hybridization probe, Probe B, which is unlabeled. If target sequence AB is a full-length 25-mer, it is expected that the signal intensity ratio of the labeled to the non-labeled probes immobilized on the solid support would be equal to 1:1 as both Probes A and B can equally hybridize to the target sequence, resulting in 50% of Probe A and 50% of Probe B hybridized to the target sequence. The hybridization can be schematically illustrated below:

| | |
|---|---|
| Probe A: | ACGTACGTAGGGGGGGA |
| Target sequence: | ACGTACGTAGGGGGGGACGTACGTA |
| Probe B: | AGGGGGGGACGTACGTA |

However, if AB is only a 20-mer with the sequence of ACGTACGTAGGGGGGGACGT, with the last five nucleotides missing due to early termination, the ratio between Probe A and Probe B hybridized to the target sequence would not be 1:1. This is because Probe A may still bind strongly to the target probe as sequence A contains all the nucleotides that match those in the sequence of the target (see the scheme below)

| | |
|---|---|
| Probe A: | ACGTACGTAGGGGGGGA |
| Target sequence: | ACGTACGTAGGGGGGGACGT |
| Probe B: | AGGGGGGGACGTACGTA |

However, Probe B would not hybridize as strongly to the target probe because only 12 out of the 17 nucleotides of Probe B can match the nucleotides in the target sequence. Therefore, Probe B would not effectively compete with Probe A in its binding to the target sequence and more non-labeled Probe B would be displaced by labeled Probe A. As a result, the intensity signal ratio of the labeled probe A to the non-labeled probe B would be greater than 1:1. It could be about 2:1, 3:1, 4:1, 5:1, 6:1 and so on, depending on the actual length of the synthesized target probe, the shorter the target sequence of AB, the higher the ratio of the labeled Probe A to the non labeled probe B.

What is claimed:

1. A method for determining the length of a target probe, comprising
(a) designing a first hybridization probe comprising a nucleic acid sequence, a portion of which overlaps with the nucleic acid sequence of a second hybridization probe;
(b) designing a second hybridization probe comprising a nucleic acid sequence, a portion of which overlaps with the nucleic acid sequence of the first hybridization probe;
(c) designing a target probe comprising the nucleic sequences of both the first and second hybridization probes or either of the first and second hybridization probe and affixing the target probe to a solid support;
(d) labeling one of the first and second hybridization probes, but not both, and contacting simultaneously the first and second probes to the target probe; and,
(e) detecting and quantifying the signal intensity ratio between the labeled and non-labeled probes, whereby said ratio indicating whether the target probe synthesis has reached full length.

2. The method of claim 1, wherein said first hybridization probe comprises a nucleic acid sequence represented by the formula:
X-Y, wherein X and Y each represent a portion of the probe;
wherein said second hybridization probe comprises a nucleic acid sequence represented by the formula:
Y-X, wherein X and Y have the same meaning above; and
wherein said target probe comprises a nucleic acid sequence represented by the general formula: X-Y-X or Y-X-Y, wherein X and Y have the same meaning above.

3. The method of claim 2, wherein said target probe further comprises a nucleic acid sequence represented by the general formula:
U-X-Y, U-Y-X, X-Y-U or Y-X-U, wherein U represents a non-matching sequence and X and Y have the same meaning as in claim 2.

4. The method of claim 1, wherein said target probe has reduced length compared to full length.

5. The method of claim 1, wherein said solid support is selected from a group consisting of porous substrates, non-porous substrates, three-dimensional surfaces, beads and planar surfaces.

6. The method of claim 5, wherein said solid support is made from materials selected from a group consisting of glass, polymers, plastics, metals, and silicon.

7. The method of claim 1, wherein said first and second probe nucleic acid sequences each has a functional length of up to about 25 nucleotides.

8. The method of claim 7, wherein said first and second probe nucleic acid sequences each has a functional length of up to about 17 nucleotides.

9. The method of claim 1, wherein said target probe nucleic acid sequences has a functional length of up to about 50 nucleotides.

10. The method of claim 9, wherein said target probe nucleic acid sequences has a functional length of up to about 25 nucleotides.

11. The method of claim 1, wherein said first, second and target probe nucleic acid sequences are selected from the group consisting of DNA, RNA, and mixtures of DNA, and RNA.

12. The method of claim 1 wherein the detectable labels are each independently selected from the group consisting of a radioisotope, a fluorescent molecule, a chemiluminescent molecule, an antibody and an enzymatically modifiable substrate, the modified enzymatic substrate being detectable.

13. A method for determining the length of a target probe, comprising
(a) providing a first hybridization probe comprising a nucleic acid sequence, a portion of which overlaps with the nucleic acid sequence of a second hybridization probe;
(b) providing a second hybridization probe comprising a nucleic acid sequence, a portion of which overlaps with the nucleic acid sequence of the first hybridization probe;
(c) providing a target probe comprising the nucleic sequences of at least part of the first and second hybridization probes;
(d) incubating said first and second probes with the target probe; and
(e) detecting the signal intensity ratio between the first and second probes, whereby said ratio indicates the length of the target probe.

14. A method of claim 13, wherein one of the first and second hybridization probes is labeled.

15. The method of claim 13, wherein said first hybridization probe comprises a nucleic acid sequence represented by the formula:
X-Y, wherein X and Y each represent a portion of the probe;
wherein said second hybridization probe comprises a nucleic acid sequence represented by the formula:
Y-X, wherein X and Y have the same meaning above; and
wherein said target probe comprises a nucleic acid sequence represented by the general formula: X-Y-X or Y-X-Y, wherein X and Y have the same meaning above.

16. The method of claim 15, wherein said target probe further comprises a nucleic acid sequence represented by the general formula:
U-X-Y, U-Y-X, X-Y-U or Y-X-U, wherein U represents a non-matching sequence and X and Y have the same meaning as in claim 2.

17. The method of claim 13, wherein said target probe has reduced length compared to full length.

18. The method of claim 13, wherein said solid support is selected from a group consisting of porous substrates, non-porous substrates, three-dimensional surfaces, beads and planar surfaces.

19. The method of claim 18, wherein said solid support is made from materials selected from a group consisting of glass, polymers, plastics, metals, and silicon.

20. The method of claim 13, wherein said first and second probe nucleic acid sequences each has a functional length of up to about 25 nucleotides.

21. The method of claim 20, wherein said first and second probe nucleic acid sequences each has a functional length of up to about 17 nucleotides.

22. The method of claim 13, wherein said target probe nucleic acid sequences has a functional length of up to about 50 nucleotides.

23. The method of claim 22, wherein said target probe nucleic acid sequences has a functional length of up to about 25 nucleotides.

24. The method of claim 13, wherein said first, second and target probe nucleic acid sequences are selected from the group consisting of DNA, RNA, and mixtures of DNA, and RNA.

25. The method of claim 13 wherein the detectable labels are each independently selected from the group consisting of a radioisotope, a fluorescent molecule, a chemiluminescent molecule, an antibody and an enzymatically modifiable substrate, the modified enzymatic substrate being detectable.
